(19) United States
(12) Patent Application Publication
(10) Pub. No.: US 2025/0177714 A1
(43) Pub. Date: Jun. 5, 2025

(54) Tattoo Machine

(71) Applicant: FK Irons Inc., Doral, FL (US)

(72) Inventor: Fernando Alberto Diaz, Miami, FL (US)

(73) Assignee: FK Irons Inc., Doral, FL (US)

(21) Appl. No.: 18/963,940

(22) Filed: Nov. 29, 2024

Related U.S. Application Data
(60) Provisional application No. 63/605,255, filed on Dec. 1, 2023.

(51) Int. Cl. A61M 37/00

(52) U.S. Cl. CPC A61M 37/0076; A61M 2205/8206

(57) ABSTRACT

A tattoo machine with an anti-roll feature is disclosed. The tattoo machine includes a tattoo grip section in which a tattoo motor is house, a battery housing that detachably couples to the grip section, and a locking cap that detachably couples to the battery housing. The tattoo grip section defines a longitudinal axis along a length of the tattoo grip section. The locking cap has a plurality of integrated protrusions that extend radially relative to the longitudinal axis farther than the battery housing exterior surface.

### CROSS REFERENCE TO RELATED APPLICATIONS

This application claims the priority benefit of Provisional Application No. 63/605,255 filed on Dec. 1, 2023, which is incorporated by reference in its entirety.

### FIELD OF THE APPLICATION

The present application relates to tattoo, permanent makeup, and micropigmentation machines.

### BACKGROUND

Tattoo machines, commonly referred to as tattoo pens, are used to apply tattoo ink, permanent makeup and pigments to a person's body and skin. A tattoo pen body is typically cylindrically shaped for ease of the tattoo artist's grip and comfort when tattooing a customer. Tattoo pens with batteries allow for a user to freely orientate the tattoo pen for the best orientation for drawing on what can be uneven surfaces. Tattoo pens can have removable batteries. The tattoo pen provides an artist with optimal functionality and comfort when applying tattoos. Such functionality includes being free to tattoo customers both inside a traditional studio and other locations.

### SUMMARY

The tattoo machine comprises a tattoo grip section in which a tattoo motor is housed and defining a longitudinal axis along a length of the tattoo grip section; a battery housing that detachably couples to the grip section and has a battery housing exterior surface; and a locking cap that detachably couples to the battery housing and has a plurality of integrated protrusions that extend radially relative to the longitudinal axis farther than the battery housing exterior surface.

In an embodiment of the tattoo machine, the locking cap comprises a proximal first ring, a middle second ring, and a distal convex portion. The proximal first ring is fixedly attached to a proximal side of the middle second ring and the middle second ring is fixedly attached to a proximal side of the distal convex portion. The distal convex portion includes recesses and a flat surface on a distal side of the distal convex portion.

In an embodiment of the tattoo machine, the outer diameter of the middle second ring is larger than an outer diameter of the proximal first ring.

In an embodiment of the tattoo machine, the proximal first ring includes tabs on an exterior radial surface. The tabs are received by notches in a cavity on the distal end of the battery housing. The locking cap is secured to the battery housing by placing the tabs of the proximal first ring within the notches on the distal end of the battery housing and rotating the locking cap in either a clockwise or counterclockwise direction. The cavity includes stops located a distance from the notches, which abut against the tabs when the locking cap is rotated in either direction.

In an embodiment of the tattoo machine, the plurality of integrated protrusions are located on an exterior radial surface of the middle second ring. The plurality of integrated protrusions prevent the tattoo machine from rolling when the tattoo machine is placed on a flat surface, wherein the protrusions project radially outward from an exterior radial surface of the locking cap.

In an embodiment of the tattoo machine, each of the plurality of integrated protrusions comprises angled surfaces, wherein the angled surfaces comprise a first surface, a second surface, a third surface, a fourth surface, a fifth surface, and a sixth surface. The first and sixth surfaces are acutely angled from the exterior radial surface of the locking cap, wherein the first and sixth surfaces form a transitional area between the protrusion and the exterior radial surface of the locking cap. The second and fifth surfaces are less acutely angled than the first and sixth surfaces, wherein the second and fifth surfaces form shoulders of the protrusion. The third and fourth surfaces form an apex of the protrusion.

In an embodiment of the tattoo machine, the angled surfaces include textured surfaces to provide additional grip to a user when turning the locking cap. The textured surfaces include knurling or cross-hatching patterns.

In an embodiment of the tattoo machine, the exterior surface of the locking cap is rubberized to provide additional grip to a user when turning the locking cap.

### DESCRIPTION OF DRAWINGS

For a more complete understanding of the present disclosure and its features and advantages, reference is now made to the following description, taken in conjunction with the accompanying drawings, in which:

FIG. 1 is a perspective view of the tattoo machine.

FIG. 2 is a side view of the tattoo machine.

FIG. 3 is an exploded view of the distal end of the tattoo machine.

FIG. 4 is a perspective exploded view of the distal end of the tattoo machine.

FIG. 5 is a top view of the distal end of the tattoo machine.

FIG. 6 is a side view of the distal end of the tattoo machine.

FIG. 7 is another perspective of a side view of the distal end of the tattoo machine.

FIG. 8 is an exploded view of the locking cap according to an embodiment of the tattoo machine.

### DETAILED DESCRIPTION

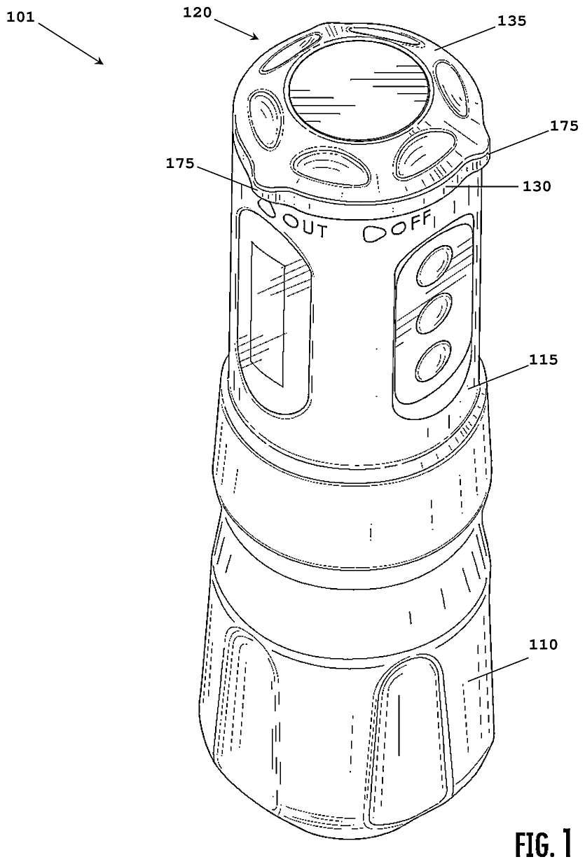

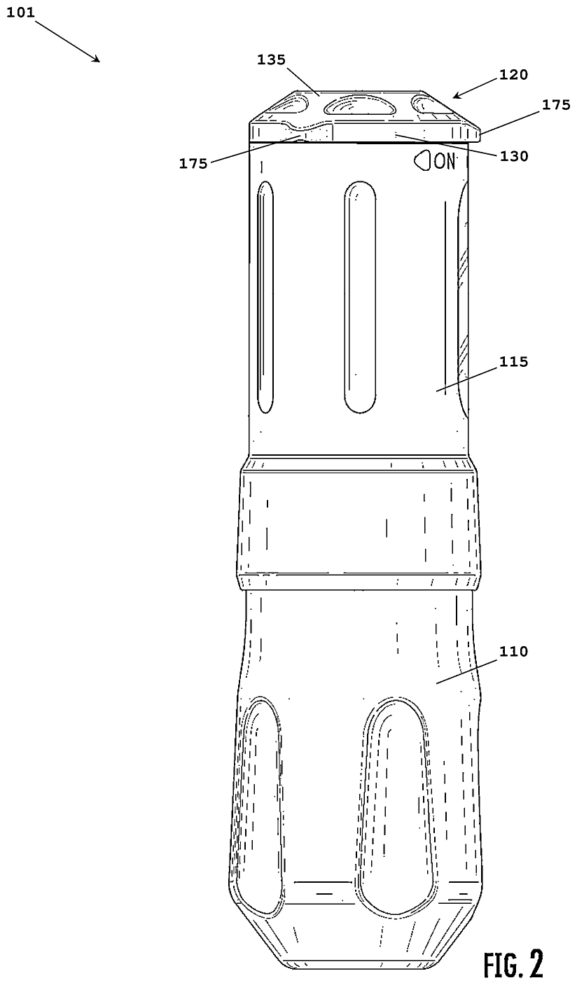

FIGS. 1 and 2 illustrate a tattoo machine 100 according to some embodiments. The tattoo machine can include a permanent makeup machine, a micropigmentation machine and/or a needling machine. The tattoo machine 100 may include a cylindrical housing 105, a motor, display, and a battery. The cylindrical housing 105 may include a grip section 110 at the work end where a needle applies ink, a battery housing 115, and a battery locking cap 120 opposite the work end. The motor may be housed within a the interior of the grip section 110. The battery may be housed within a portion of the interior of the battery housing 115.

In some embodiments, the battery housing 115 may detachably couple to the distal end of the grip section 110 via a threaded attachment or magnetic coupling. The proximal end of locking cap 120 may detachably couple to the distal end of the battery housing 115. In other arrangements, the locking cap 120 can be non-detachably coupled to the battery housing 115 or integral with the battery housing 115 such that the locking cap 120 and the battery housing 115 are unibody.

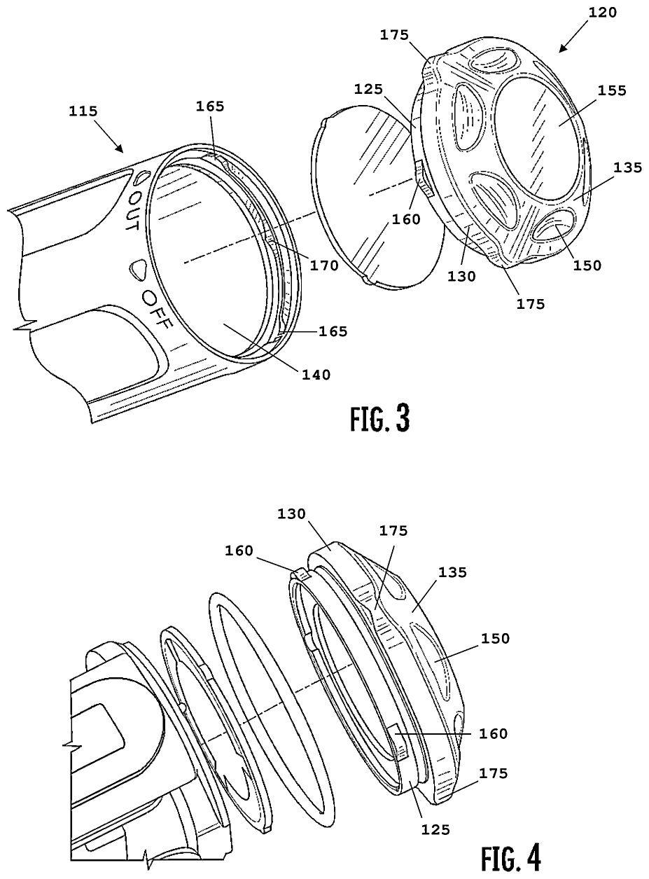

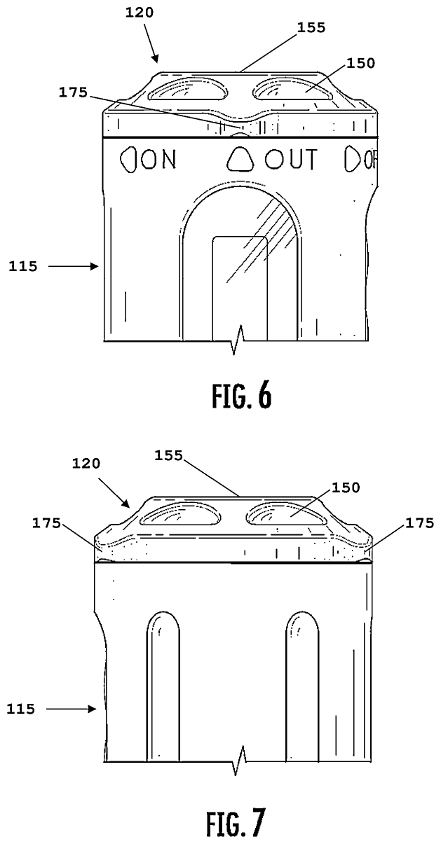

FIGS. 3 through 7 illustrate a distal end of tattoo machine 100. FIGS. 3 and 4 illustrate an exploded view of the distal end of tattoo machine 100. The locking cap 115 may include a proximal first ring 125, a middle second ring 130, and distal convex portion 135. The distal end of battery housing 115 may include a cavity 140 for receiving a proximal first ring 125. The proximal first ring 125 may have an outer diameter that is approximately equal to the inner diameter of the cavity 140. The middle second ring 130 may have a larger outer diameter than the proximal first ring 125. The distal convex portion 135 may include concentrically arranged recesses 150 and a flat surface 155. A user may use recesses 150 to rotate locking cap 120 by placing their fingertips within the recesses 150 and rotating the locking cap 120 in either a clockwise direction or a counter-clockwise direction.

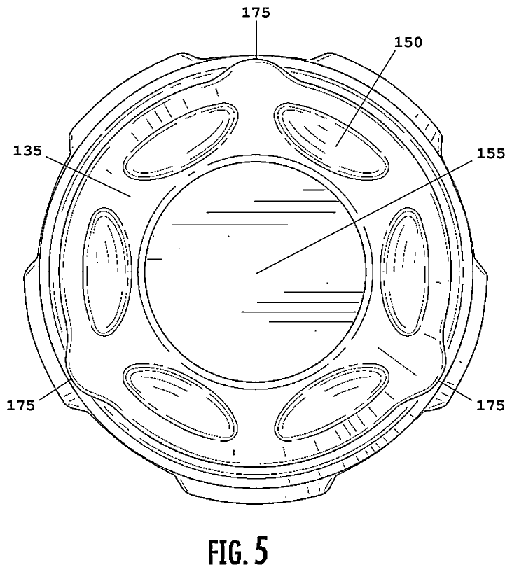

FIG. 5 illustrates a top-down view of the distal end of tattoo machine 100. From this perspective, the orientation of the integral protrusions 175 can be seen relative to cut-outs and lateral extensions of the battery housing 115. The cut-outs are concentrically positioned approximately 720 off set from an adjacent cut-out. The lateral extensions are concentrically positioned approximately 720 off set from an adjacent lateral extension. The integral protrusions 175 are concentrically positioned approximately 1200 off set from an adjacent integral protrusion.

In some embodiments, the locking cap 120 may be detachably secured to the battery housing 115. The proximal first ring 125 may have three tabs 160 on that are received by three notches 170 inside the cavity 140 on the distal end of battery housing 115. The tabs 160 and notches 165 may be oriented with respect to each other such that locking cap 120 cannot be attached to the battery housing 115 unless each tab 160 is aligned with each of the corresponding notches 165. Once the tabs 160 are properly aligned with notches 165, a user may rotate locking cap 120 in either a clockwise or counter-clockwise direction to secure the locking cap 120 to the battery housing 115. The cavity 140 may also include two stops 170, which may be placed a distance clockwise and counter-clockwise from notches 165. As the user rotates the locking cap 120 in either direction, tabs 160 will abut against either stop 170 preventing the locking cap 120 from rotating further.

In some embodiments, the proximal first ring 125 may include a groove. The groove receives an O-ring, which provides a water-tight seal between the distal end of battery housing 115 and locking cap 120.

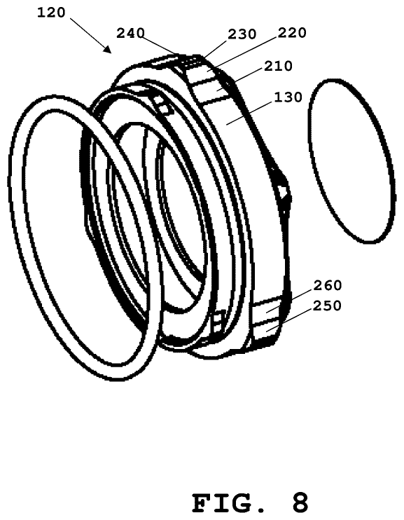

FIG. 8 illustrates the anti-roll structural arrangement according to some embodiments. The anti-roll structural arrangement may include a plurality of integral protrusions 175 on the outer surface of the locking cap 120 where integral protrusions 175 are unibody with the locking cap 120. In another arrangement, a single integral protrusion 175 may be included. In one embodiment, the anti-roll integral protrusions 175 may be located on the exterior radial surface of the middle second ring 130 and integral with the middle second ring 130 and cap 120. The integral protrusions 175 extend radially outward from a longitudinal central axis of the tattoo machine 100. The integral protrusions 175 may extend beyond the exterior surface of the battery housing 115 a length that is 2% greater than the circumference of the outer surface of the battery housing 115. Other arrangements are integral protrusions 175 extending beyond the exterior surface of the battery housing 115 by 1%-10% greater than the circumference of the outer surface of the battery housing 115. In another arrangement, the integral protrusions 175 may extend, 0.3 cm to 1.5 cm beyond the circumference of the outer surface of the battery housing 115. In some arrangements, the integral protrusions 175 may extend a uniform distance. In other arrangement, the integral protrusions 175 may extend non-uniform distances.

The integral protrusions 175 may include three integral protrusions 175 concentrically positioned approximately 120° off set from an adjacent integral protrusion 175. One, two, four or other number of integral protrusions 175 can be provided. In some arrangements, each integral protrusion 175 can be equidistant from each another integral protrusion 175. In arrangements, each integral protrusion 175 can be located circumferentially on cap 120 a distance from a tab 310 such that turning the cap 120 via integral protrusion 175 a quarter turn secures the cap 120 to the tattoo machine 100.

The plurality of integral protrusions 175 may extend radially outward from the exterior radial surface of the locking cap 120. Each protrusion 175 may be made of angled surfaces 210-260. The angled surfaces may include a first surface 210, a second surface 220, a third surface 230, a fourth surface 240, a fifth surface 250, and a sixth surface 260. The first surface 210 and sixth surface 260 may be acutely angled from the exterior radial surface of the locking cap 120 to form a transitional area between the protrusion 175 and the exterior radial surface of the locking cap 120. The second surface 220 and fifth surface 250 may be less acutely angled than the first surface 210 and the sixth surface 260 to form the shoulders of the protrusion 175. The third surface 230 and fourth surface 240 form the apex of the protrusion 175. In one embodiment, the third surface 230 and fourth surface 240 may be more acutely angled than the second surface 220 and fifth surfaces 250 such that the third surface 230 and fourth surface 240 are beveled to form a rounded apex. In another embodiment, the third surface 230 and fourth surface 240 may be less acutely angled than the second surface 220 and fifth surface 250 such that the third surface 230 and fourth surface 240 appear to form a sharp apex.

The locking cap 120 may be made from plastic, metal, rubber, or a combination thereof. In one embodiment, the locking cap 120 may be formed from a thermoplastic material using an injection molding process. In another embodiment, the locking cap 120 may be formed from metal using a mill, lathe, or water jet cutting machine or other process to form protrusions 175 that are integrated with cap 120. In some embodiments, the outer surface of the locking cap 120 may be treated to provide additional grip to a user turning the locking cap. For example, the entire exterior surface of the locking cap 120 may be rubberized. In another example, the protrusions 175 of the anti-roll structural arrangement may be textured using patterns such as knurling or cross-hatching.

The integral protrusions 175 may prevent the tattoo machine 100 from rolling when placed on a flat surface, such as a table or stand. The integral protrusions 175 may also provide additional grip to allow a user to turn the locking cap 120. The integral protrusions 175 may also allow the user to place the tattoo machine 100 on a flat surface such that the display 140 is facing upwards towards the user while the tattoo machine 100 is stationary on the flat surface. Because the cap 120 rotates relative to the battery housing 115, an integral protrusion 175 or plurality of integral protrusions 175 can be rotated by a user to orientate the tattoo machine 115, and thus display 140, for easy view by user. An example of such a use is a user rotating cap 120 via an integral protrusion 175 or plurality of integral protrusions 175 such that the display screen of tattoo device 100 is viewable while the tattoo machine 100 is resting on a table.

The illustrations of arrangements described herein are intended to provide a general understanding of the structure of various embodiments, and they are not intended to serve as a complete description of all the elements and features of apparatus and systems that might make use of the structures described herein. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure not be limited to the particular arrangement(s) disclosed as the best mode contemplated for carrying out this invention, but that the invention will include all embodiments and arrangements falling within the scope of the appended claims.

The foregoing is provided for purposes of illustrating, explaining, and describing embodiments of this invention. Modifications and adaptations to these embodiments will be apparent to those skilled in the art and may be made without departing from the scope or spirit of this invention. Upon reviewing the aforementioned embodiments, it would be evident to an artisan with ordinary skill in the art that said embodiments can be modified, reduced, or enhanced without departing from the scope and spirit of the claims described below. Although some of the drawings illustrate a number of operations/steps in a particular order, operations which are not order-dependent may be reordered and other operations may be combined or broken out. While some reordering or other groupings are specifically mentioned, others will be apparent to those of ordinary skill in the art and so do not present an exhaustive list of alternatives.

1. A tattoo machine, comprising;
a tattoo grip section in which a tattoo motor is housed and defining a longitudinal axis along a length of the tattoo grip section;
a battery housing that detachably couples to the grip section and has a battery housing exterior surface;
a locking cap that detachably couples to the battery housing and has a plurality of integrated protrusions that extend radially relative to the longitudinal axis farther than the battery housing exterior surface.

2. The tattoo machine of claim 1, wherein the locking cap further comprises a proximal first ring, a middle second ring, and a distal convex portion, wherein the proximal first ring is fixedly attached to a proximal side of the middle second ring, wherein the middle second ring is fixedly attached to a proximal side of the distal convex portion.

3. The tattoo machine of claim 2, wherein an outer diameter of the middle second ring is larger than an outer diameter of the proximal first ring.

4. The tattoo machine of claim 2, wherein the distal convex portion includes recesses and a flat surface on a distal side of the distal convex portion.

5. The tattoo machine of claim 2, wherein the distal end of the battery housing further includes a cavity for receiving the proximal first ring of the locking cap.

6. The tattoo machine of claim 5, wherein the proximal first ring includes tabs on an exterior radial surface, wherein the tabs are received by notches in the cavity on the distal end of the battery housing, wherein the locking cap is secured to the battery housing by placing the tabs of the proximal first ring within the notches on the distal end of the battery housing and rotating the locking cap in either a clockwise or counterclockwise direction.

7. The tattoo machine of claim 6, wherein the cavity further includes stops located a distance from the notches, wherein the tabs abut against the stops when the locking cap is rotated in either direction.

8. The tattoo machine of claim 1, wherein the plurality of integrated protrusions are located on an exterior radial surface of the middle second ring.

9. The tattoo machine of claim 1, wherein the plurality of integrated protrusions prevent the tattoo machine from rolling when the tattoo machine is placed on a flat surface, wherein the protrusions project radially outward from an exterior radial surface of the locking cap.

10. The tattoo machine of claim 1, wherein each of the plurality of integrated protrusions comprises angled surfaces, wherein the angled surfaces comprise a first surface, a second surface, a third surface, a fourth surface, a fifth surface, and a sixth surface.

11. The tattoo machine of claim 10, wherein the first and sixth surfaces are acutely angled from the exterior radial surface of the locking cap, wherein the first and sixth surfaces form a transitional area between the protrusion and the exterior radial surface of the locking cap.

12. The tattoo machine of claim 10, wherein the second and fifth surfaces are less acutely angled than the first and sixth surfaces, wherein the second and fifth surfaces form shoulders of the protrusion.

13. The tattoo machine of claim 10, wherein the third and fourth surfaces form an apex of the protrusion.

14. The tattoo machine of claim 10, wherein the angled surfaces include textured surfaces to provide additional grip to a user when turning the locking cap.

15. The tattoo machine of claim 14, wherein the textured surfaces include knurling or cross-hatching patterns.

16. The tattoo machine of claim 1, wherein an exterior surface of the locking cap is rubberized to provide additional grip to a user when turning the locking cap.

17. The tattoo machine of claim 1, wherein the locking cap is made out of plastic, metal, rubber or a combination thereof.

18. The tattoo machine of claim 19, wherein the locking cap is machines from a sheet or block of metal using a mill, lathe, or water jet cutting machine.

19. A tattoo device, comprising:
a tattoo grip section in which a tattoo motor is housed and defining a longitudinal axis along a length of the tattoo grip section, wherein the tattoo grip section detachably couples to a battery housing;
a locking cap that detachably couples to the battery housing and has a plurality of integrated protrusions that extend radially relative to the longitudinal axis farther than the battery housing exterior surface.
